(19) United States
(12) Patent Application Publication
(10) Pub. No.: US 2017/0189465 A1
(43) Pub. Date: Jul. 6, 2017

(54) Anti Hallucination Drug

(71) Applicant: Kracie Pharma, Ltd., Minato-ku, Tokyo (JP)

(72) Inventors: Hisayoshi Norimoto, Takaoka-shi, Toyama (JP); Chiaki Murayama, Takaoka-shi, Toyama (JP); Seiwa Michihara, Takaoka-shi, Toyama (JP)

(73) Assignee: Kracie Pharma, Ltd., Minato-ku, Tokyo (JP)

(21) Appl. No.: 15/314,754

(22) PCT Filed: Mar. 26, 2015
(86) PCT No.: PCT/JP2015/059471

§ 371 Date: Nov. 29, 2016

(30) Foreign Application Priority Data
May 29, 2014 (JP) 2014-111175
Sep. 19, 2014 (JP) 2014-190603

(51) Int. Cl. A61K 36/284; G01N 33/15; G01N 33/50; A61K 31/343; A61K 31/015

(52) U.S. Cl. CPC A61K 36/284; A61K 31/343; A61K 31/015; G01N 33/50; G01N 33/15

(57) ABSTRACT

With the object of providing a novel anti-hallucination agent capable of suppressing, treating or preventing a hallucinatory symptom, the anti-hallucination agent of the invention contains an Atractylodes-derived crude drug as an active ingredient or contains a compound represented by the following formula (Ia), a stereoisomer thereof or a salt thereof as an active ingredient: in the formula, the double lines consisting of a solid line and a broken line represent a single bond or a double bond; R₁ represents a hydrogen atom or a hydroxyl group, with the proviso that R₁ does not exist when the carbon atom to which R₁ is attached is linked to one of the neighboring carbon atoms through a double bond; R₂ represents a hydrogen atom or is combined with R₃ to represent a cyclic group which may have a substituent; and R₃ represents a hydrogen atom or an alkyl group having 1 to 6 carbon atoms which may have a substituent or is combined with R₂ to represent a cyclic group which may have a substituent.

### TECHNICAL FIELD

The present invention relates to an anti-hallucination agent for suppressing, treating or preventing a hallucinatory symptom.

### BACKGROUND ART

The term “hallucination” is one of medical terms, especially one of psychiatric terms, and refers to a perception without an object, namely a symptom of the experience of a sensation in the absence of actual external input. Hallucinations include for example auditory hallucinations, which are hallucinations of the auditory sensation, visual hallucinations, which are hallucinations of the visual sensation, and the like.

Causes of hallucinations are abuse of drugs such as hallucinogens, stimulant drugs and cannabis, mental disorders such as schizophrenia, posttraumatic stress disorder (PTSD), behavioral and psychological symptoms of dementia and the like.

The mechanisms of hallucinations have not been clearly known, but it is believed that neurotransmitters such as dopamine and serotonin are involved. For example, phenylalkylamine-based drugs such as mescaline, indoleamine-based drugs such as psilocybin and LSD and the like are known as hallucinogenic drugs having an agonistic action on serotonin receptors. The hallucinogenic action is believed to depend on 5-HT_{2A} receptor, of serotonin receptors.

(+/−) 2,5-dimethoxy-4-iodoamphetamine (DOI), which is an agonist of the serotonin receptor 5-HT_{2A}, is also known as a hallucinogen. It has been reported that specific head-twitching response (HTR) is induced when DOI is administered to mice and rats (NPL 1). Moreover, a correlation between the drug-induced head-twitching response in an animal and a hallucinatory symptom in human has been observed, and it has been found that a clinically effective agent for treating a hallucination reduces the head-twitching response in an animal (NPL 2).

A piperazine derivative having a 5-HT₂ receptor antagonistic action and a 5-HT_{1A} receptor agonistic action is disclosed in PTL 1 as an agent for treating or preventing a psychoneurosis including hallucinations. Moreover, PTL 2 discloses an agent for treating a mental disorder accompanying cerebrovascular accident which contains, as an essential component, a compound that can block both serotonin 2 receptor and dopamine D2 receptor.

### CITATION LIST

### Patent Literature

PTL 1: JP-A-2000-204040
PTL 2: JP-A-2001-316264

### Non Patent Literature

NPL 1: Darmani NA1, Martin B R, Pandey U, Glennon R A. Pharmacol. Biochem. Behav. 36, 901-906, 1990.
NPL 2: Corne S J, Pickering R W. Psychopharmacologia. 11, 65-78, 1967.

### SUMMARY OF INVENTION

### Technical Problem

A drug capable of more effectively and safely suppressing a hallucinatory symptom which is caused by various causes as described above is desired. Thus, an object of the invention is to provide a novel anti-hallucination agent capable of suppressing, treating or preventing a hallucinatory symptom.

### Solution to Problem

To solve the problems, the present inventors have administered extracts of atractylodes rhizome (Byakujutsu) and atractylodes lancea rhizome (Sojutsu), which are derived from Atractylodes (Jutsu) plants, to mice for a certain period and then induced hallucination in the mice using a hallucinogen, DOI. As a result, the inventors have found that the head-twitching response in the mice, which is an indicator of hallucination, was suppressed. The inventors have also administered atractylone, atractylenolide atractylenolide III and β-eudesmol, which are compounds contained in atractylodes rhizome and atractylodes lancea rhizome, to mice for a certain period and found that these compounds have an activity of suppressing the head-twitching response in mice. Based on the findings, the inventors have completed the invention.

That is, the invention provides an anti-hallucination agent containing an Atractylodes-derived crude drug as an active ingredient.

The invention also provides an anti-hallucination agent containing a compound represented by the following formula (Ia), a stereoisomer thereof or a salt of the compound or the stereoisomer as an active ingredient:

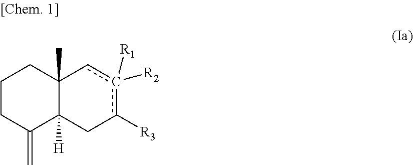

wherein, the double lines consisting of a solid line and a broken line represent a single bond or a double bond; R₁ represents a hydrogen atom or a hydroxyl group, providing that R₁ does not exist when the carbon atom to which R₁ is attached is linked to one of the neighboring carbon atoms through a double bond; R₂ represents a hydrogen atom or is combined with R₃ to represent a cyclic group which may have a substituent; and R₃ represents a hydrogen atom or an alkyl group having 1 to 6 carbon atoms which may have a substituent or is combined with R₂ to represent a cyclic group which may have a substituent.

The invention also provides an anti-hallucination agent, wherein the compound is represented by the following formula (Ib).

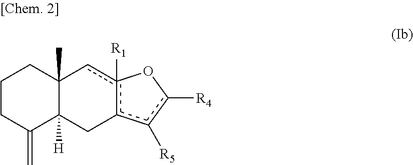

wherein the double lines consisting of a solid line and a broken line represent a single bond or a double bond; R₁ represents a hydrogen atom or a hydroxyl group, with the proviso that R₁ does not exist when the carbon atom to which R₁ is attached is linked to one of the neighboring carbon atoms through a double bond; R₄ represents a hydrogen atom or an oxo group; and R₅ represents a hydrogen atom or an alkyl group having 1 to 4 carbon atoms which may have a substituent.

The invention also provides an anti-hallucination agent, wherein the compound is represented by the following formula (II), (III), (IV) or (V).

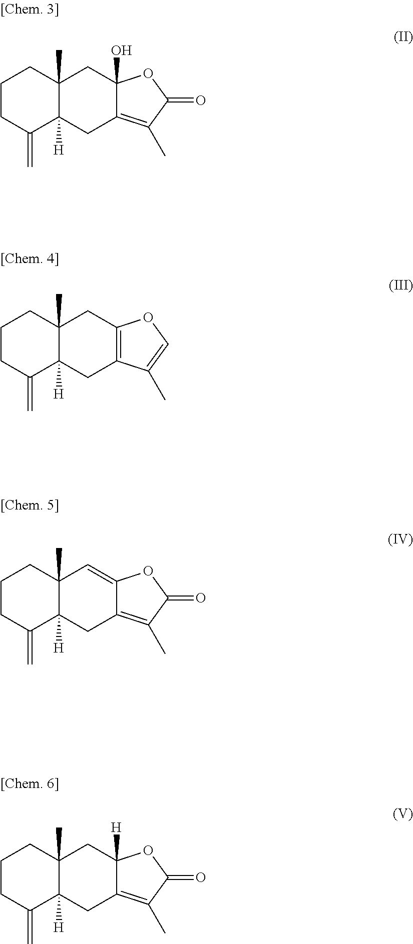 [SMILES: C=C1CCC[C@]2(C)C[C@]3(O)OC(=O)C(C)=C3C[C@@H]12 ; C=C1CCC[C@]2(C)Cc3occ(C)c3C[C@@H]12 ; C=C1CCC[C@]2(C)C=C3OC(=O)C(C)=C3C[C@@H]12 ; C=C1CCC[C@]2(C)C[C@@H]3OC(=O)C(C)=C3C[C@@H]12]

The invention also provides the anti-hallucination agent, wherein R₃ represents an alkyl group having 1 to 6 carbon atoms which has a hydroxyl group.

The invention also provides the anti-hallucination agent, wherein R₃ is a 2-hydroxyisopropyl group.

The invention also provides a method for screening a compound having an anti-hallucination action, including

a step of providing a compound represented by the following formula (Ia) or a derivative thereof as a test compound,

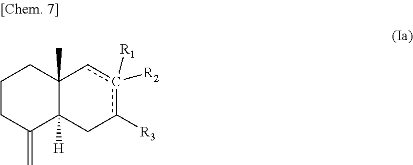

wherein, the double lines consisting of a solid line and a broken line represent a single bond or a double bond; R₁ represents a hydrogen atom or a hydroxyl group, with the proviso that R₁ does not exist when the carbon atom to which R₁ is attached is linked to one of the neighboring carbon atoms through a double bond; R₂ represents a hydrogen atom or is combined with R₃ to represent a cyclic group which may have a substituent; and R₃ represents a hydrogen atom or an alkyl group having 1 to 6 carbon atoms which may have a substituent or is combined with R₂ to represent a cyclic group which may have a substituent, and

a step of determining whether or not the test compound has an anti-hallucination activity.

### Advantageous Effects of Invention

Because the anti-hallucination agent according to the invention contains an Atractylodes-derived crude drug or a compound having an anti-hallucination action as an active ingredient, the anti-hallucination agent can be used for a drug for suppressing, treating or preventing a hallucinatory symptom. For example, the anti-hallucination agent according to the invention can be used for a drug for suppressing, treating or preventing a hallucinatory symptom caused by abuse of a drug such as hallucinogens, stimulant drugs and cannabis, a mental disorder such as schizophrenia, posttraumatic stress disorder (PTSD) or dementia.

### BRIEF DESCRIPTION OF DRAWINGS

FIG. 1 A graph showing the effects of an atractylodes rhizome extract and an atractylodes lancea rhizome extract on the hallucinogen-induced head-twitching response in mice.

FIG. 2 A graph showing the effects of atractylenolide III and β-eudesmol on the hallucinogen-induced head-twitching response in mice.

FIG. 3 A graph showing the effects of atractylone on the hallucinogen-induced head-twitching response in mice.

FIG. 4 A graph showing the effects of atractylenolide II on the hallucinogen-induced head-twitching response in mice.

FIG. 5 A graph showing the effects of atractylenolide I on the hallucinogen-induced head-twitching response in mice.

FIG. 6 A graph showing the effects of single application of each compound on the hallucinogen-induced head-twitching response in mice.

FIG. 7 A graph showing the effects of single application of atractylenolide III on the hallucinogen-induced head-twitching response in mice.

### DESCRIPTION OF EMBODIMENTS

### (Anti-Hallucination Agent)

The anti-hallucination agent of the invention is a drug for suppressing, treating or preventing a hallucinatory symptom.

The term “hallucination” in this description refers to a perception without an object, namely a symptom of the experience of a sensation in the absence of actual external input. Hallucinations include auditory hallucinations, visual hallucinations, olfactory hallucinations, gustatory hallucinations, tactile hallucinations and the like.

The anti-hallucination agent of the invention is effective at least against a hallucinatory symptom caused by abnormality of the serotonergic nervous system and effective for example against a hallucinatory symptom caused by a hallucinogen such as a serotonin receptor agonist. Moreover, the anti-hallucination agent of the invention can be used for example for suppressing, treating or preventing a hallucinatory symptom caused by abuse of a drug such as hallucinogens, stimulant drugs and cannabis, a mental disorder such as schizophrenia, posttraumatic stress disorder (PTSD) and dementia.

The anti-hallucination agent of the invention contains an Atractylodes (Jutsu)-derived crude drug as an active ingredient. Atractylodes plants refer to the genus Atractylodes of the family Compositae and include for example Atractylodes macrocephala Koidzumi, Atractylodes lancea De Candolle and Atractylodes chinensis Koidzumi. Examples of the Atractylodes-derived crude drug include atractylodes rhizome, atractylodes lancea rhizome and the like. For example, the anti-hallucination agent of the invention can contain one of or both of atractylodes rhizome and atractylodes lancea rhizome.

The raw material of atractylodes rhizome (Byakujutsu) is generally the rhizome of Atractylodes japonica Koidzumi ex Kitamura (Wa-byakujutsu), the rhizome of Atractylodes macrocephala Koidzumi (Kara-byakujutsu) or the rhizome of another plant of the genus.

The raw material of atractylodes lancea rhizome (Sojutsu) is generally the rhizome of Atractylodes lancea De Candolle, Atractylodes chinensis Koidzumi, a hybrid thereof or another plant of the genus (Compositae).

The above-described part of a plant is preferably used, but not limited as the raw material plant of a crude drug used in the invention. One, or two or more parts selected from flower, spike, peel, fruit, stem/twig, leaf, branch, branch/leaf, trunk, bark, rhizome, root bark, root, seed, whole herb and the like of any of the above-described plants can be used as the raw material of a crude drug.

The crude drug used in the invention may be the raw material plant itself or may be an extract of the raw material. The term “extract” in the invention includes an extract obtained by extracting from the raw material using a solvent, a solution obtained after compressing the raw material, an extract obtained by compressing the raw material and subsequently extracting from the residue using a solvent, a dry solid product obtained by drying and solidifying such an extract or the solution obtained after compression and the like.

The crude drug used in the invention can be produced by a known method. The crude drug used in the invention can be produced for example through extraction at the normal temperature or extraction under heating, using an extractant such as water, an alcohol including methanol and ethanol or a mixed solvent thereof. If required, the extraction may be conducted under reduced pressure or under pressure. The obtained extract may be used directly or may be used after being dried and solidified through concentration or freeze-drying.

The invention also provides an anti-hallucination agent containing a compound represented by the following formula (Ia), a stereoisomer thereof or a salt of the compound or the stereoisomer as an active ingredient.

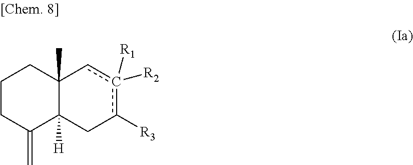

In the above formula (Ia), the double lines consisting of a solid line and a broken line represent a single bond or a double bond. In other words, the carbon atom to which R₁ and R₂ are attached may be linked to the neighboring carbon atoms through single bonds or may be linked to one of the neighboring carbon atoms through a double bond.

R₁ represents a hydrogen atom or a hydroxyl group. However, R₁ does not exist when the carbon atom to which R₁ is attached is linked to one of the neighboring carbon atoms through a double bond.

R₂ represents a hydrogen atom or is combined with R₃ to represent a cyclic group which may have a substituent.

R₃ represents a hydrogen atom or an alkyl group having 1 to 6 carbon atoms which may have a substituent or is combined with R₂ to represent a cyclic group which may have a substituent.

In this description, examples of the substituent include an alkyl group having 1 to 4 carbon atoms which may have a substituent, an alkoxy group having 1 to 4 carbon atoms which may have a substituent, halogen atoms, hydroxyl group, oxo group, carboxyl group, amino group, nitro group, cyano group and the like. Examples of the alkyl group having 1 to 4 carbon atoms include methyl group, ethyl group, n-propyl group, i-propyl group, n-butyl group, i-butyl group, t-butyl group and the like. Examples of the alkoxy group having 1 to 4 carbon atoms include methoxy group, ethoxy group, n-propoxy group, i-propoxy group, n-butoxy group, i-butoxy group, t-butoxy group and the like. The halogen atoms include fluorine, chlorine, bromine and iodine. Examples of the substituents which the alkyl group and the alkoxy group as the substituents may have include halogen atoms, hydroxyl group, oxo group, carboxyl group, amino group, nitro group, cyano group and the like.

In this description, examples of the alkyl group having 1 to 6 carbon atoms which may be represented by R₃ include linear or branched alkyl groups such as methyl group, ethyl group, n-propyl group, i-propyl group, n-butyl group, i-butyl group, t-butyl group, n-pentyl group, i-pentyl group, neopentyl group, hexyl group, 1-methylpropyl group, 1-ethylpropyl group, 1-methylbutyl group and 2-methylbutyl group. The alkyl group is preferably methyl group, ethyl group, n-propyl group, i-propyl group, 2-methyl-1-propyl group, t-butyl group or the like.

When R₂ and R₃ are combined and represent a cyclic group which may have a substituent, the cyclic group can be for example a carbocyclic group or a heterocyclic group. The cyclic group can be a cyclic group of any size and can be for example a 3- to 7-membered ring, preferably a 5-membered ring or a 6-membered ring. Examples of the cyclic group include furan ring, thiophene ring, pyrrole ring, imidazole ring, pyrrolidine ring, benzene ring, pyran ring, pyridine ring, pyrimidine ring, pyrazine ring, piperidine ring, piperazine ring, morpholine ring and the like.

For example, R₂ and R₃ may be combined and represent a furan ring which may have a substituent. The anti-hallucination agent of the invention may contain for example a compound represented by the following formula (Ib) as the compound represented by the above formula (Ia).

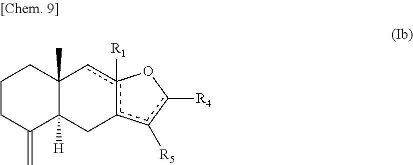

In the above formula (Ib), the double lines consisting of a solid line and a broken line represent a single bond or a double bond. R₁ is as described above in the description of R₁ of the above formula (Ia). R₄ represents a hydrogen atom or an oxo group. R₅ represents a hydrogen atom or an alkyl group having 1 to 4 carbon atoms which may have a substituent. Examples of the alkyl group having 1 to 4 carbon atoms which may be represented by R₅ include methyl group, ethyl group, n-propyl group, i-propyl group, n-butyl group, i-butyl group, t-butyl group and the like. R₅ is preferably a methyl group.

For example, the anti-hallucination agent of the invention may contain the compound represented by the following formula (II), atractylenolide III, as the compound represented by the above formula (Ib). Atractylenolide III is one of the components contained in a crude drug such as atractylodes rhizome and atractylodes lancea rhizome and may be contained in the anti-hallucination agent as a crude drug such as atractylodes rhizome or atractylodes lancea rhizome.

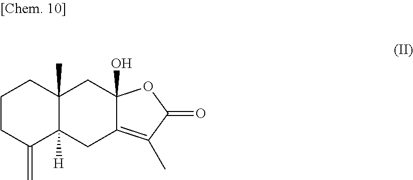 [SMILES: C=C1CCC[C@]2(C)C[C@]3(O)OC(=O)C(C)=C3C[C@@H]12]

Moreover, the anti-hallucination agent of the invention may contain for example a compound represented by anyone of the following formulae (III) to (VI), a stereoisomer or a tautomer thereof or a salt of the compound or the isomer as the compound represented by the above formula (Ib). The compound represented by the following formula (III) is atractylone, and the compound represented by the following formula (IV) is atractylenolide I. The compound represented by the following formula (V) is atractylenolide II, and the compound represented by the following formula (VI) is 8-epiasterolide. These compounds are also components contained in a crude drug such as atractylodes rhizome and atractylodes lancea rhizome and may be contained in the anti-hallucination agent as a crude drug such as atractylodes rhizome or atractylodes lancea rhizome.

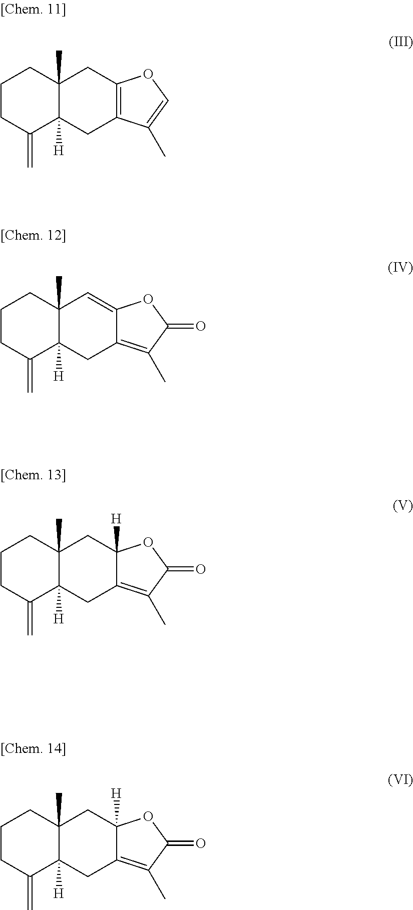 [SMILES: C=C1CCC[C@]2(C)Cc3occ(C)c3C[C@@H]12 ; C=C1CCC[C@]2(C)C=C3OC(=O)C(C)=C3C[C@@H]12 ; C=C1CCC[C@]2(C)C[C@@H]3OC(=O)C(C)=C3C[C@@H]12 ; C=C1CCC[C@]2(C)C[C@H]3OC(=O)C(C)=C3C[C@@H]12]

Furthermore, the anti-hallucination agent of the invention may contain a compound represented by the following formula (I) as the compound represented by the above formula (Ia).

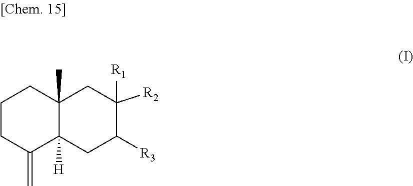

In the above formula (I), R₁ represents a hydrogen atom or a hydroxyl group; R₂ represents a hydrogen atom or is combined with R₃ to represent a cyclic group which may have a substituent; and R₃ represents a hydrogen atom or an alkyl group having 1 to 6 carbon atoms which may have a substituent or is combined with R₂ to represent a cyclic group which may have a substituent.

In the above formula (Ia) or (I), R₃ may represent an alkyl group having 1 to 6 carbon atoms which has a hydroxyl group and may be for example 2-hydroxyisopropyl group. In this case, R₁ and R₂ may each represent a hydrogen atom. For example, the anti-hallucination agent of the invention may contain the compound represented by the following formula (VII), β-eudesmol, as the compound represented by the above formula (Ia) or (I). β-Eudesmol is one of the components contained in a crude drug such as atractylodes rhizome and atractylodes lancea rhizome and may be contained in the anti-hallucination agent as a crude drug such as atractylodes rhizome or atractylodes lancea rhizome.

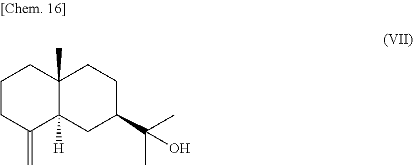 [SMILES: C=C1CCC[C@]2(C)CC[C@@H](C(C)(C)O)C[C@@H]12]

The anti-hallucination agent of the invention may contain one, two or more compounds represented by the above formula (Ia), stereoisomers thereof or salts of the compounds or the stereoisomers. For example, the anti-hallucination agent of the invention may contain at least one compound selected from the group consisting of atractylone, atractylenolide I, atractylenolide II, atractylenolide III, 8-epiasterolide and β-eudesmol, a stereoisomer or a tautomer thereof or a salt of the compound or the isomer as an active ingredient.

The above-described compounds can be obtained through isolation from an Atractylodes extract and purification. Alternatively, the above-described compounds can be obtained from a compound which is isolated and purified from an Atractylodes extract as a starting compound, through modification such as introduction or substitution of a functional group or a substituent, oxidation, reduction and substitution or addition of an atom by various methods known to one skilled in the art.

For example, the above-described compounds can be obtained by fractionating an extract of atractylodes rhizome or atractylodes lancea rhizome by chromatography or the like once, twice or more as described in detail in the Examples. The fractionation can be conducted for example by silica gel column chromatography using hexane, ethyl acetate, acetone, methanol, hexane/ethyl acetate, hexane/acetone or the like as the mobile phase.

The compound of the invention can be synthesized from tetralone, which is commercially available, as a starting material through Oppenauer oxidation and Wittig olefination as described for example in Bagal S K, Adlington R M, Baldwin J E, Marquez R., J Org Chem. 69, 9100-9108, 2004 and Bagal SK, Adlington R M, Baldwin J E, Marquez R, Cowley A., Org Lett. 5, 3049-3052, 2003. As tetralone, for example, α-tetralone, which is represented by the following formula (VIII), 6-methoxy-1-tetralone and the like can be used.

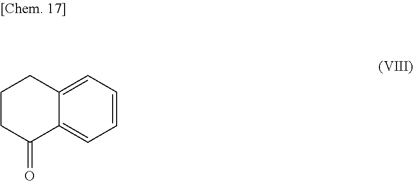 [SMILES: O=C1CCCc2ccccc21]

In an example method for synthesizing the compound of the invention from tetralone, for example, tetralone is first reduced and converted into the corresponding alcohol, and then the alcohol is subjected to the conditions for Birch reduction to partially reduce the benzene ring, thereby generating the corresponding enol ether. Then, a TMS enol ether intermediate compound is generated through Oppenauer oxidation and addition of a cuprate, and then the intermediate compound is converted into the corresponding ketone through fluoride-mediated desilylation. Then, the carbonyl group can be converted into a methylene group through Wittig olefination. One skilled in the art can easily synthesize various compounds included in the above formula (I) by appropriately modifying this method.

The anti-hallucination agent of the invention can further contain any component. For example, the anti-hallucination agent of the invention may further contain a crude drug other than atractylodes rhizome and atractylodes lancea rhizome, which are described above. Moreover, the anti-hallucination agent of the invention can be provided in the form further containing a diluent, a binder, a disintegrating agent, a lubricant and a coloring agent which are pharmaceutically acceptable and the like.

Examples of the diluent used for the anti-hallucination agent include lactose, glucose, white soft sugar, mannitol, dextrin, potato starch, corn starch, calcium carbonate, calcium phosphate, calcium sulfate, crystalline cellulose and the like.

Examples of the binder include starch, gelatin, syrup, tragacanth gum, polyvinyl alcohol, polyvinyl ether, polyvinylpyrrolidone, hydroxypropylcellulose, methylcellulose, ethylcellulose, carboxymethylcellulose and the like.

Examples of the disintegrating agent include starch, agar, gelatin powder, crystalline cellulose, calcium carbonate, sodium hydrogen carbonate, sodium alginate, sodium carboxymethylcellulose, calcium carboxymethylcellulose and the like.

Examples of the lubricant include magnesium stearate, hydrogenated vegetable oil, talc, macrogol and the like. Moreover, as the coloring agent, any coloring agent which has been approved to be added to pharmaceutical products can be used.

In addition, the anti-hallucination agent, if required, may be coated with one or more layers of white soft sugar, gelatin, refined shellac, gelatin, glycerin, sorbitol, ethylcellulose, hydroxypropylcellulose, hydroxypropylmethylcellulose, polyvinylpyrrolidone, phthalic acid cellulose acetate, hydroxypropylmethylcellulose phthalate, methyl methacrylate, a methacrylic acid polymer or the like.

Moreover, if required, a pH-controller, a buffering agent, a stabilizer, a solubilizing agent and the like may be added.

In addition, the anti-hallucination agent can be provided as a pharmaceutical preparation in any form. For example, the anti-hallucination agent as a pharmaceutical preparation for oral administration can be a tablet such as sugar coated tablets, buccal tablets, coating tablets and chewable tablets, a troche, a pill, powder, a capsule including soft capsules, granules, a suspension, an emulsion, syrup including dry syrup or a liquid such as elixirs.

In addition, for parenteral administration, the anti-hallucination agent can be a pharmaceutical preparation for administration such as intravenous injection, subcutaneous injection, intraperitoneal injection, intramuscular injection, transdermal administration, nasal administration, transpulmonary administration, enteral administration, buccal administration and transmucosal administration. For example, the anti-hallucination agent can be an injection, a transdermal absorbing tape, an aerosol preparation, a suppository or the like.

The crude drug or the compound used for the anti-hallucination agent of the invention can be provided at any dose and can be provided for example at a dose which is generally taken as a Kampo medicine. For example, the crude drug or the compound can be provided in such a manner that the total dose of the crude drug becomes 1 mg to 500 mg per day.

The anti-hallucination agent of the invention can be provided in a form which is administered in multiple doses. The administration in multiple doses means that the anti-hallucination agent is administered twice or more at a predetermined dosing interval. Regarding the dosing interval and the number of administrations, any interval and any number which are effective for suppressing, treating or preventing the hallucinatory action in the subject can be selected. For example, the dosing interval can be several hours, a day, several days, a week or the like. Moreover, the number of administrations can be twice or more, preferably five times or more, more preferably 10 times or more. The anti-hallucination agent of the invention may be administered everyday continuously for example for two days or longer, preferably for five days or longer, more preferably for 10 days or longer. As will be shown in the Examples described below, the anti-hallucination agent of the invention can suppress, treat or prevent a hallucinatory symptom more certainly when the anti-hallucination agent is administered in multiple doses.

The anti-hallucination agent of the invention can be in the form of a pharmaceutical product, a quasi-drug, food, drink or the like for suppressing, treating or preventing a hallucinatory symptom.

The invention also provides a method for suppressing, treating or preventing a hallucinatory symptom including a step of administering an effective dose of an Atractylodes-derived crude drug to a subject. Furthermore, the invention provides a method for suppressing, treating or preventing a hallucinatory symptom including a step of administering an effective dose of a compound represented by the above formula (Ia), a stereoisomer thereof or a salt of the compound or the stereoisomer to a subject.

In this description, the “effective dose” means the dose which is effective for suppressing, treating or preventing a hallucinatory symptom of the subject of administration. The “subject” of administration also includes human, non-human mammals and non-mammals.

The way of administration of the anti-hallucination agent includes oral administration and parenteral administration, such as intravenous injection, subcutaneous injection, intraperitoneal injection, intramuscular injection, transdermal administration, nasal administration, transpulmonary administration, enteral administration, buccal administration and transmucosal administration. The anti-hallucination agent of the invention as a pharmaceutical preparation for oral administration can be administered in the form of a tablet such as sugar coated tablets, buccal tablets, coating tablets and chewable tablets, a troche, a pill, powder, a capsule including soft capsules, granules, a suspension, an emulsion, syrup including dry syrup or a liquid such as elixirs. In addition, the anti-hallucination agent of the invention as a preparation for parenteral administration can be administered in the form of for example an injection, a transdermal absorbing tape, an aerosol preparation, a suppository or the like.

The crude drug or the compound used for the anti-hallucination agent which is administered in the method for suppressing, treating or preventing a hallucinatory symptom of the invention can be administered at any dose and can be administered for example at a dose which is generally taken as a Kampo medicine, once or multiple times. For example, the anti-hallucination agent used in the method of the invention can be administered in such a manner that the total dose of the crude drug becomes 1 mg to 500 mg per day.

### (Screening Method)

The invention further provides a method for screening a compound having an anti-hallucination action. The method of the invention includes a step of providing a compound represented by the above formula (Ia) or a derivative thereof as a test compound and a step of determining whether or not the test compound has an anti-hallucination activity.

In this description, the “derivative” of a compound is a compound obtained through modification such as introduction or substitution of a functional group or a substituent, oxidation, reduction or substitution or addition of an atom to an extent which does not largely modify the structure or the properties of the compound as the base.

The compound represented by the above formula (Ia) can be obtained by the above-described method. The derivative of the compound represented by the above formula (Ia) can be obtained from the compound represented by the above formula (Ia) as a starting compound through modification such as introduction or substitution of a functional group or a substituent, oxidation, reduction and substitution or addition of an atom by various methods known to one skilled in the art.

In the method of the invention, the test compound may be a compound represented by the above formula (Ia) or a derivative thereof and is not particularly limited. However, the test compound may be for example a compound selected from the group consisting of atractylone, atractylenolide I, atractylenolide II, atractylenolide III, 8-epiasterolide, β-eudesmol and derivatives thereof.

For the method for determining whether or not the test compound has an anti-hallucination activity, any known method can be used. For example, the test compound can be determined to have an anti-hallucination activity when a hallucinatory symptom is caused in an animal by administering a hallucinogen to the animal and then the hallucinatory symptom, for example the head-twitching response, in the animal is suppressed significantly by the administration of the test compound. For example, the step of determining whether or not the test compound has an anti-hallucination activity may include a step of administering the test compound to an animal, a step of administering a hallucinogen to the animal and a step of counting head-twitches of the animal.

The hallucinogen can be any hallucinogen, and for example, LSD (lysegic acid diethylamine), phencyclidine (PCP), cannabis, thinners, psilocybin, mescaline, 3,4-methylenedioxymethamphetamine (MDMA), (+/−) 2,5-dimethoxy-4-iodoamphetamine (DOI) and the like can be used.

### EXAMPLES

The embodiments of the invention are explained in further detail with the Examples below, but the invention is not limited to the Examples below.

### Example 1

### [Preparation of Atractylodes Rhizome Extract]

At room temperature, 600 g of a crude drug atractylodes rhizome (Atractylodes japonica) was immersed in 10 times the amount of n-hexane for two days, and extraction was conducted twice through filtration. Then, the filtrate of n-hexane was concentrated under reduced pressure, and about 27.3 g of an extract (an atractylodes rhizome extract) was obtained (yield of 4.55%).

[Isolation of Compounds from Atractylodes Rhizome Extract and Purification]

The atractylodes rhizome extract (20 g) was subjected to various types of column chromatography, and the compounds atractylone, atractylenolide I, atractylenolide II, atractylenolide III and 8-epiasterolide were obtained.

Specifically, first, 20 g of the atractylodes rhizome extract was applied to a silica gel 60 (63 to 200 μm) column (i.d. 90×370 mm) and eluted with hexane-ethyl acetate (100:0) (fraction A), hexane-ethyl acetate (100:5) (fraction B), hexane-ethyl acetate (100:10) (fraction C), hexane-ethyl acetate (100:20) (fraction D), hexane-ethyl acetate (100:50) (fraction E), hexane-ethyl acetate (0:100) (fraction F) and methanol (fraction G) in this order. Thus, seven fractions (A to G) were obtained.

Then, the fraction B was applied to a VersaPak column attached with a silica cartridge and eluted repeatedly with hexane-ethyl acetate (in the order of ratios 100:0→0:100). The eluate was recrystallized, and the compounds atractylone (726.4 mg) and atractylenolide I (20.1 mg) were purified as white needle-like crystals.

Moreover, the fraction C was applied to a VersaPak column attached with a silica cartridge and eluted repeatedly with hexane-ethyl acetate (in the order of ratios 100:0-÷0:100). The eluate was recrystallized, and the compounds atractylone (200.2 mg), atractylenolide II (259.6 mg) and 8-epiasterolide (167.9 mg) were purified as white needle-like crystals.

Furthermore, the fraction E was applied to a VersaPak column attached with a silica cartridge and eluted repeatedly with hexane-acetone (in the order of ratios 100:0→0:100). The eluate was recrystallized, and the compound atractylenolide III (164.1 mg) was purified as white needle-like crystals.

### Example 2

### [Preparation of Atractylodes Lancea Rhizome Extract]

To 50 g of a crude drug atractylodes lancea rhizome, about four times the amount of water was added, and the mixture was heated with a mantle heater and refluxed for 30 minutes after the mixture was boiled, and extraction was conducted. The extract was treated with a freeze dryer, and about 10.8 g of dry powder (an atractylodes lancea rhizome extract) was obtained.

### [Preparation of β-Eudesmol]

When a crude drug atractylodes lancea rhizome is left in a refrigerator at a low temperature (4° C.), white silk thread-like crystals are generated on the surface of the crude drug. The crystals are crystals of essential oil mainly containing β-eudesmol. β-Eudesmol can be easily extracted with a low-polarity solvent such as hexane.

Moreover, β-eudesmol is commercially available, and commercial β-eudesmol (Lot. WEG2032, Wako Pure Chemical Industries, Ltd.) was used in Test 3 described below.

[Analysis of Main Components in Crude Drugs using HPLC]

The main components of atractylodes rhizome and atractylodes lancea rhizome were analyzed using HPLC under the following conditions.

1) Conditions of HPLC Analysis

The apparatus was LC-10ATvp manufactured by Shimadu Corporation (the detector was SPD-10A UV-VIS); the column was a Purospher (registered trademark) STAP RP-18e reverse-phase column (5 μm, 4 mm i.d.×252 mmm, Merck KGaA, Germany); the column temperature was 40° C.; the mobile phase was A: acetonitrile/B: 0.05% TFA; the flow rate was 1 ml/min (45% acetonitrile 15 minutes, 53% acetonitrile 15 minutes, 68% acetonitrile 15 minutes, 83% acetonitrile 15 minutes); and the detection wavelength was 190-230 nm.

2) Absolute Calibration Curve:
atractylenolide III: y=3122.8x−12454 R²=0.9999
atractylenolide II: y=4897.5x−21305 R²=0.9999
β_eudesmol: y=625.7x+206282 R²=0.9997

### (Results of Analysis)

The results of the analysis are shown in the table below.

TABLE 1

| | Content (mg/g crude drug) | | |
|---|---|---|---|
| | Atractylenolide | Atractylenolide | |
| | III | II | β_Eudesmol |
| Atractylodes | 1.63 | 0.54 | 5.92 |
| rhizome | | | |
| Atractylodes | 0.16 | 0.04 | 11.0 |
| lancea rhizome | | | |

### [Test 1: Production of Hallucination Model Mouse]

Four-week-old ICR male mice (17-19 g) were purchased from Japan SLC, Inc. and subjected to the experiment after an acclimatization period of a week. The animals were kept at a room temperature of 23±2° C. at a humidity of 55±10% in a 12-hour cycle of turning on the light at 8:00 and turning off the light at 20:00. The experimental animals were allowed to drink water and eat freely during the period of the experiment.

In a physiological saline solution, (+/−) 2,5-dimethoxy-4-iodoamphetamine (DOI: sigma Lot 0.012M4812V), which is a hallucinogen, was dissolved. Immediately after intraperitoneally injecting the solution to the mice at 1 mg/kg, the mice were moved to a breeding cage without a floor mat. After five minutes, the numbers of head-twitches of the mice (used as an indicator of a hallucinatory symptom) were counted for five minutes (Egashira et al., Repeated administration of Yokukansan inhibits DOI-induced head-twitch response and decreases expression of 5-hydroxytryptamine (5-HT) 2A receptors in the prefrontal cortex., Prog Neuropsychopharmacol Biol Psychiatry., 32, 1516-1520, 2008). In this regard, to exclude the influence of external noise or the like on the behavior of the mice, the head-twitches were counted in a soundproof chamber.

As a result, it was confirmed that the numbers of head-twitches of the DOI-administered mice were higher than those of normal mice. Therefore, it could be confirmed that a hallucination model mouse can be produced by this method.

### [Test 2: Anti-Hallucination Actions of Atractylodes Rhizome Extract and Atractylodes Lancea Rhizome Extract]

The anti-hallucination actions of the atractylodes rhizome extract and the atractylodes lancea rhizome extract were examined. In this test, the atractylodes rhizome extract and the atractylodes lancea rhizome extract prepared in Examples 1 and 2 were used.

Similar ICR male mice to those used in Test 1 were randomly divided into eight groups including a normal group, a control group and six test groups. A 0.5% CMC-Na solution was orally administered to the normal group and the control group once a day for 14 days, and the atractylodes rhizome extract at different doses (20, 100 and 500 mg/kg) and the atractylodes lancea rhizome extract at different doses (20, 100 and 500 mg/kg), which were each prepared using a 0.5% CMC-Na solution, were orally administered to the six test groups once a day for 14 days. One hour after the completion of the final oral administration, 1 mg/kg of DOI was intraperitoneally injected to the mice of the control group and the test groups. Then, the head-twitches of the mice were counted in a similar manner as in Test 1. The results of the measurement were tested by the Dunnett's test using one-way analysis of variance for the comparison between multiple groups. Here, when p<0.05, it was determined that the result attained statistical significance.

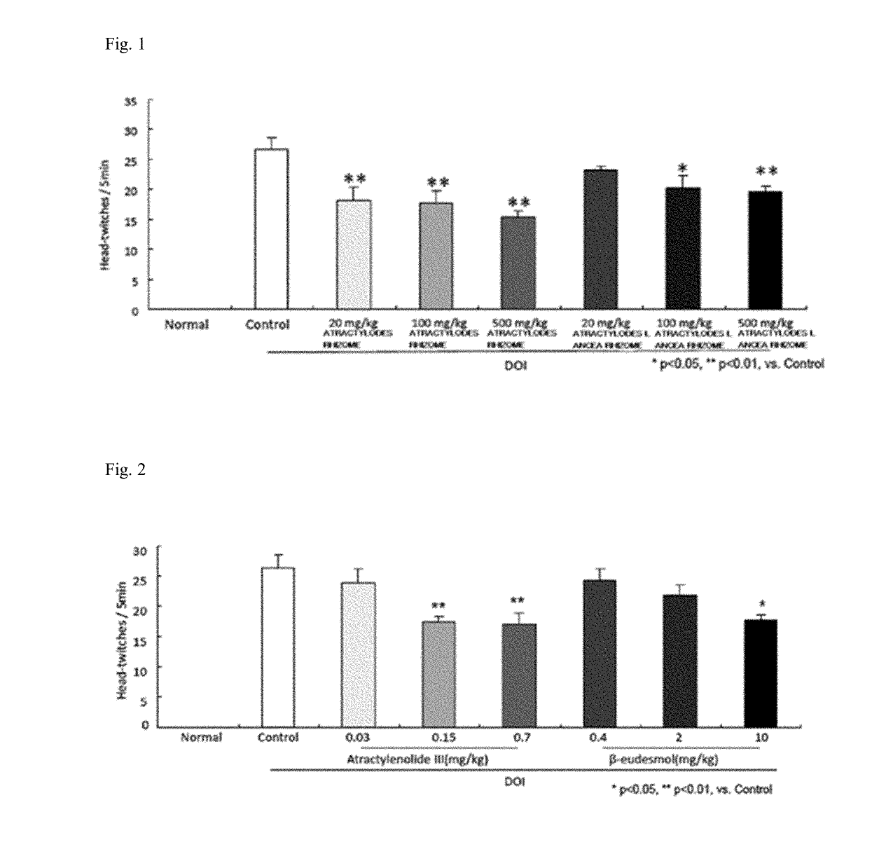

FIG. 1 is a graph showing the effects of the atractylodes rhizome extract and the atractylodes lancea rhizome extract on the hallucinogen-induced head-twitching response in mice. The number of head-twitches decreased significantly in the test groups administered the atractylodes rhizome extract at the all doses as compared to the control group. In the test groups administered the atractylodes lancea rhizome, the number of head-twitches decreased significantly at the doses of 100 and 500 mg/kg as compared to the control group. The results showed that the atractylodes rhizome extract and the atractylodes lancea rhizome extract had an action of suppressing the hallucinogen-induced head-twitching response in mice. Therefore, it was strongly suggested that the atractylodes rhizome extract and the atractylodes lancea rhizome extract have an anti-hallucination action.

### [Test 3: Anti-Hallucination Actions of Atractylenolide III and β-Eudesmol]

The anti-hallucination actions of atractylenolide III and β-eudesmol were examined by the same method as that of Test 2 except for the points which are especially specified. In this test, atractylenolide III purified in Example 1 and commercial β-eudesmol (Lot. WEG2032, Wako Pure Chemical Industries, Ltd.) were dissolved in corn oil and used. The doses were set based on the average amounts of the compounds contained in the crude drugs and on the doses used in Test 2. Specifically, the doses of atractylenolide III were set at 0.03, 0.15 and 0.7 mg/kg, and the doses of β-eudesmol were set at 0.4, 2.0 and 10 mg/kg.

FIG. 2 is a graph showing the effects of atractylenolide III and β-eudesmol on the hallucinogen-induced head-twitching response in mice. In the atractylenolide III-administered test groups, the number of head-twitches decreased significantly at the doses of 0.15 and 0.7 mg/kg as compared to the control group. In the β-eudesmol-administered test groups, the number of head-twitches decreased significantly at the dose of 10 mg/kg as compared to the control group. The results showed that atractylenolide III and β-eudesmol had an action of suppressing the hallucinogen-induced head-twitching response in mice. Therefore, it was strongly suggested that atractylenolide III and β-eudesmol have an anti-hallucination action.

### [Test 4: Anti-Hallucination Actions of Atractylone and Atractylenolide II]

The anti-hallucination actions of atractylone and atractylenolide II were examined by the same method as that of Test 2 except for the points which are especially specified. In this test, atractylone and atractylenolide II purified in Example 1 were dissolved in corn oil and used. The doses of atractylone were set at 0.36, 1.8 and 9.0 mg/kg, and the doses of atractylenolide II were set at 0.03, 0.15 and 0.75 mg/kg.

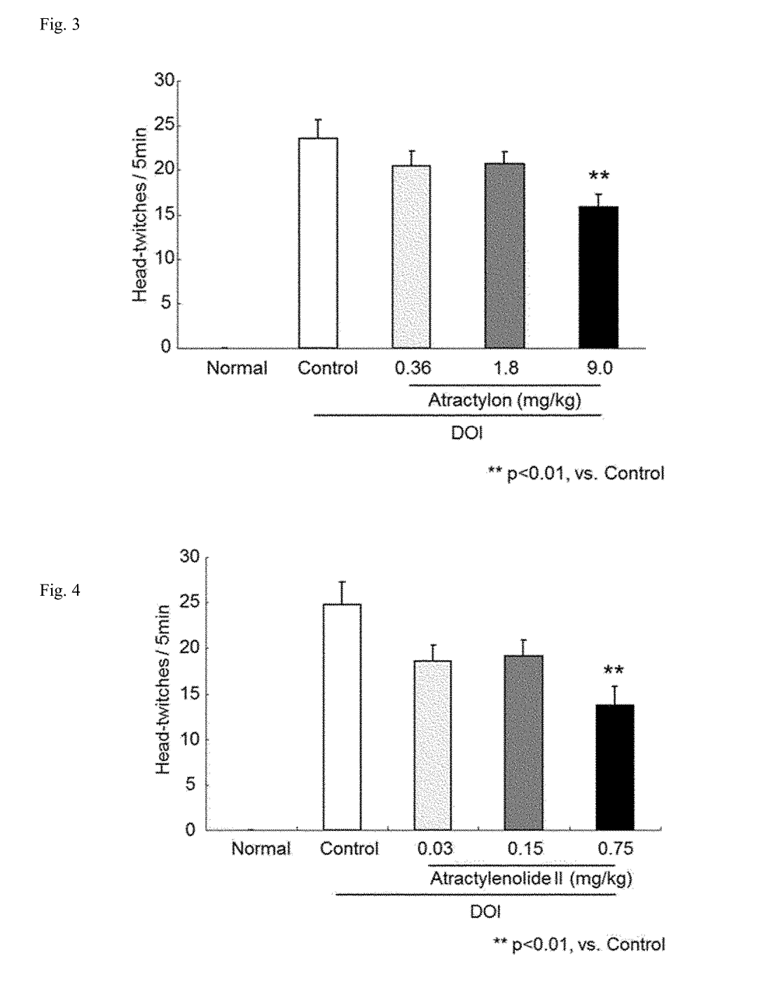

FIG. 3 is a graph showing the effects of atractylone on the hallucinogen-induced head-twitching response in mice. In the atractylone-administered test groups, the number of head-twitches decreased significantly at the dose of 9.0 mg/kg as compared to the control group. Moreover, FIG. 4 is a graph showing the effects of atractylenolide II on the hallucinogen-induced head-twitching response in mice. In the atractylenolide II-administered test groups, the number of head-twitches decreased significantly at the dose of 0.75 mg/kg as compared to the control group. The results showed that atractylone and atractylenolide II had an action of suppressing the hallucinogen-induced head-twitching response in mice. Therefore, it was strongly suggested that atractylone and atractylenolide II have an anti-hallucination action.

### [Test 5: Anti-Hallucination Action of Atractylenolide I]

The anti-hallucination action of atractylenolide I was examined by the same method as that of Test 2 except for the points which are especially specified. In this test, atractylenolide I purified in Example 1 was dissolved in corn oil and used. The doses of atractylenolide I were set at 0.03, 0.15 and 0.75 mg/kg.

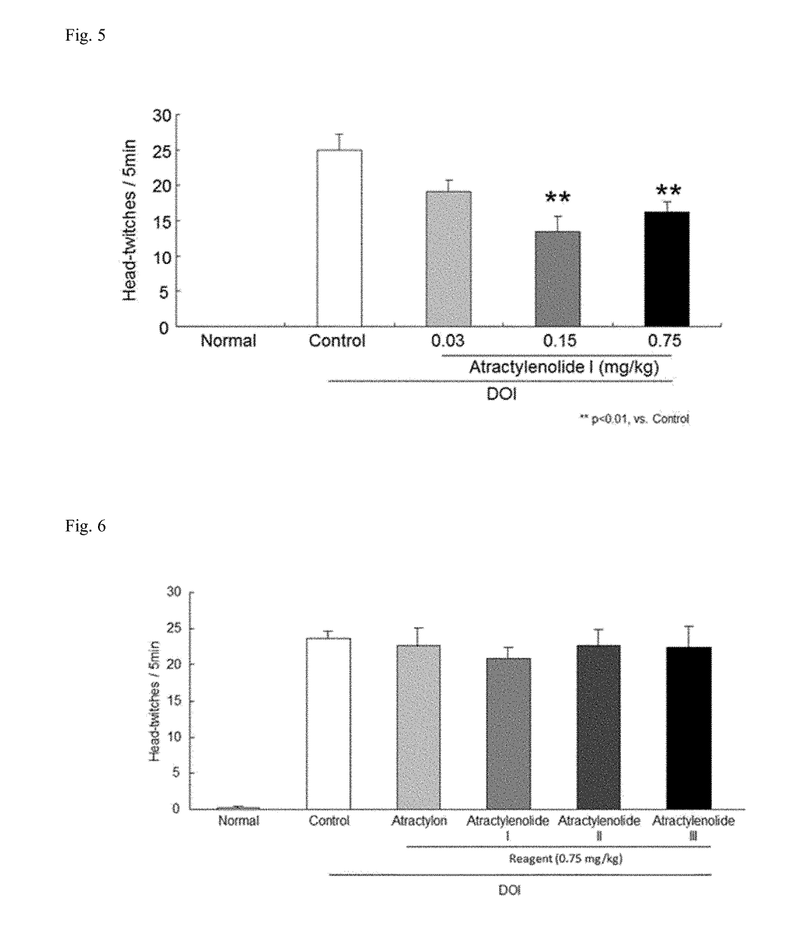

FIG. 5 is a graph showing the effects of atractylenolide I on the hallucinogen-induced head-twitching response in mice. In the atractylenolide I-administered test groups, the number of head-twitches decreased significantly at the doses of 0.15 and 0.75 mg/kg as compared to the control group. The results showed that atractylenolide I had an action of suppressing the hallucinogen-induced head-twitching response in mice. Therefore, it was strongly suggested that atractylenolide I has an anti-hallucination action.

### [Test 6: Effects of Single Application]

The anti-hallucination action of each of the compounds atractylone, atractylenolide I, atractylenolide II and atractylenolide III in the case of one oral administration only (single application) was examined. The same method as that of Test Example 2 was used except that each compound was administered once. The compounds at a dose of 0.75 mg/kg were orally administered once to the test groups. After one hour, DOI was intraperitoneally injected, and the head-twitches of the mice were counted. Also, atractylenolide III at doses of 0.03, 0.15 and 0.75 mg/kg was orally administered once to other test groups. After one hour, DOI was intraperitoneally injected, and the head-twitches of the mice were counted.

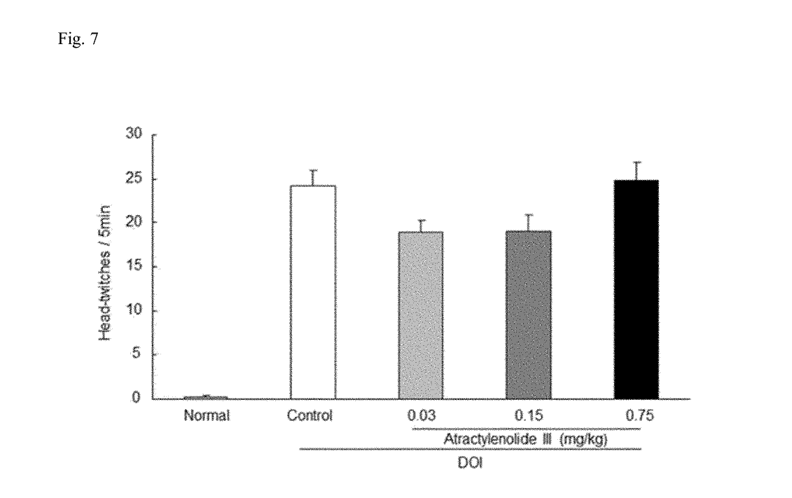

FIG. 6 is a graph showing the effects of single application of each compound on the hallucinogen-induced head-twitching response in mice. FIG. 7 is a graph showing the effects of single application of atractylenolide III on the hallucinogen-induced head-twitching response in mice. In all the compound-administered test groups, no significant difference in the number of head-twitches from that of the control group was observed. Because these compounds suppressed the head-twitching response significantly in Tests 3 to 5, in which the compounds were administered in multiple doses, it was suggested that the compounds have higher effects when being administered in multiple doses.

The specific embodiments and the Examples described in this description are merely used for clarifying the technical contents of the invention. The invention should not be construed as being limited to these specific examples, and modification is possible in the scope of the spirit and the claims of the invention.

The present application is based on a Japanese application filed on May 29, 2014 (Patent Application No. 2014-111175) and a Japanese application filed on Sep. 19, 2014 (Patent Application No. 2014-190603), which are hereby incorporated by reference in their entirety.

### INDUSTRIAL APPLICABILITY

The invention can be suitably used for a drug for suppressing, treating or preventing a hallucinatory symptom.

1. An anti-hallucination agent containing an Atractylodes-derived crude drug as an active ingredient.

2. An anti-hallucination agent containing a compound represented by the following formula (Ia), a stereoisomer thereof or a salt of the compound or the stereoisomer as an active ingredient: 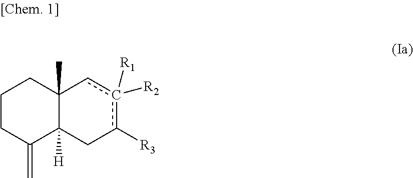
in the formula,
the double lines consisting of a solid line and a broken line represent a single bond or a double bond,
R₁ represents a hydrogen atom or a hydroxyl group, with the proviso that R₁ does not exist when the carbon atom to which R₁ is attached is linked to one of the neighboring carbon atoms through a double bond,
R₂ represents a hydrogen atom or is combined with R₃ to represent a cyclic group which may have a substituent, and
R₃ represents a hydrogen atom or an alkyl group having 1 to 6 carbon atoms which may have a substituent or is combined with R₂ to represent a cyclic group which may have a substituent.

3. The anti-hallucination agent according to claim 2, wherein the compound is represented by the following formula (Ib): 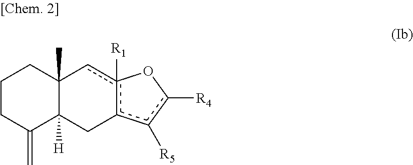
in the formula,
the double lines consisting of a solid line and a broken line represent a single bond or a double bond,
R₁ represents a hydrogen atom or a hydroxyl group, with the proviso that R₁ does not exist when the carbon atom to which R₁ is attached is linked to one of the neighboring carbon atoms through a double bond,
R₄ represents a hydrogen atom or an oxo group, and
R₅ represents a hydrogen atom or an alkyl group having 1 to 4 carbon atoms which may have a substituent.

4. The anti-hallucination agent according to claim 3, wherein the compound is represented by the following formula (II), (III), (IV) or (V):

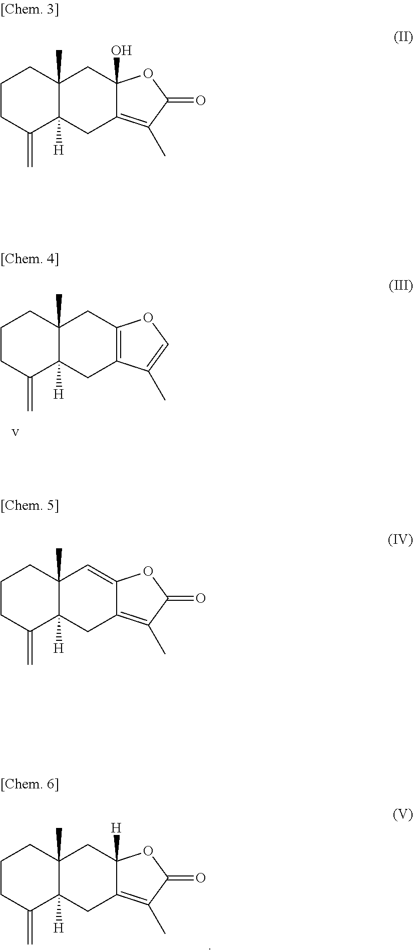 [SMILES: C=C1CCC[C@]2(C)C[C@]3(O)OC(=O)C(C)=C3C[C@@H]12 ; C=C1CCC[C@]2(C)Cc3occ(C)c3C[C@@H]12 ; C=C1CCC[C@]2(C)C=C3OC(=O)C(C)=C3C[C@@H]12 ; C=C1CCC[C@]2(C)C[C@@H]3OC(=O)C(C)=C3C[C@@H]12]

5. The anti-hallucination agent according to claim 2, wherein R₃ represents an alkyl group having 1 to 6 carbon atoms which has a hydroxyl group.

6. The anti-hallucination agent according to claim 5, wherein R₃ is a 2-hydroxyisopropyl group.

7. A method for screening a compound having an anti-hallucination action, including
a step of providing a compound represented by the following formula (Ia) or a derivative thereof as a test compound, 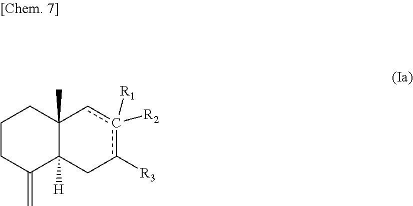
in the formula, the double lines consisting of a solid line and a broken line represent a single bond or a double bond, R₁ represents a hydrogen atom or a hydroxyl group, with the proviso that R₁ does not exist when the carbon atom to which R₁ is attached is linked to one of the neighboring carbon atoms through a double bond, R₂ represents a hydrogen atom or is combined with R₃ to represent a cyclic group which may have a substituent, and R₃ represents a hydrogen atom or an alkyl group having 1 to 6 carbon atoms which may have a substituent or is combined with R₂ to represent a cyclic group which may have a substituent, and
a step of determining whether or not the test compound has an anti-hallucination activity.
